(19) United States
(12) Patent Application Publication
(10) Pub. No.: US 2026/0327533 A1
(43) Pub. Date: Oct. 22, 2026

(54) METHODS AND MICROORGANISMS FOR PRODUCING SOUR BEER

(71) Applicant: Iowa State University Research Foundation, Inc., Ames, IA (US)

(72) Inventor: Scott Nelson, Ames, IA (US)

(21) Appl. No.: 19/644,019

(22) Filed: Apr. 10, 2026

Related U.S. Application Data
(60) Provisional application No. 63/786,693, filed on Apr. 10, 2025.

(51) Int. Cl. C12C 12/00; C12C 5/02; C12C 11/00; C12C 11/06; C12C 11/11; C12N 1/165; C12R 1/645

(52) U.S. Cl. CPC C12C 12/006; C12C 5/026; C12C 11/003; C12C 11/06; C12C 11/11; C12N 1/165; C12R 2001/645

(57) ABSTRACT

The disclosure relates to methods for producing a sour fermented beverage using Lachancea fermentati strain SWN001 and to kits comprising the strain.

### CROSS REFERENCE TO RELATED APPLICATIONS

This application claims priority to provisional application U.S. Ser. No. 63/786,693 filed Apr. 10, 2025, which is incorporated herein by reference in its entirety.

### SEQUENCE LISTING XML

The instant application contains a sequence listing, which has been submitted in XML file format by electronic submission and is hereby incorporated by reference in its entirety. The XML file, created on Apr. 2, 2026, is named P14729US01.xml and is 2,363 bytes in size.

### TECHNICAL FIELD

The present disclosure relates to compositions and methods for making sour fermented beverages and other fermentation products.

### BACKGROUND

Traditionally, sour beers are made through mixed fermentation, involving a combination of yeast and bacteria, such as Lactobacillus or Pediococcus species. This process is effective in producing the desired sourness, but it can be complex, time-consuming, and unpredictable due to the interactions between different microorganisms. Thus, there is a need in the art for yeast strains that can be used in the fermentation of sour beer.

### SUMMARY

The identification of a yeast species that is capable of lowering the pH of beer represents a significant advancement in brewing technology, particularly for the production of sour beers. A single species of yeast that can naturally acidify beer simplifies the brewing process by enabling a single-microorganism fermentation. This approach offers several advantages: it allows for more consistent and controllable sourness levels, reduces the risk of cross-contamination with other beer styles produced in the same brewery, and potentially shortens the fermentation time.

Methods of producing a fermented beverage comprising fermenting at least one carbohydrate in the presence of a Lachancea fermentati strain to produce a fermented beverage are provided. In certain embodiments, the Lachancea fermentati strain is Lachancea fermentati strain SWN001 deposited as ATCC Accession No. PTA-127803. In certain embodiments, the Lachancea fermentati strain comprises the nucleotide sequence of SEQ ID NO: 1 within the ITS region.

Biologically pure cultures of a Lachancea fermentati strain of the disclosure are provided. Kits comprising a Lachancea fermentati strain of the disclosure and one or more ingredients to produce a fermented beverage are also provided.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A-B shows the carbohydrate utilization and ethanol tolerance of Lachancea fermentati strain SWN001 (ISU-Lf #14).

FIG. 2 shows the souring capabilities of Lachancea fermentati strain SWN001 (ISU-Lf #14) compared to a commercial yeast species Lachancea thermotolerans known as Philly Sour and sold by Lallemand Brewing.

FIG. 3 shows the growth kinetics of Lachancea fermentati strain SWN001 (ISU-Lf #14) compared to the Philly Sour yeast.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the term “biologically pure culture” refers to a microbial culture that is free or essentially free of biological contamination and that has genetic uniformity such that different subcultures taken therefrom will exhibit identical or substantially identical genotypes and phenotypes. In certain embodiments, the biologically pure culture is 100% pure (i.e., all subcultures taken therefrom exhibit identical genotypes and phenotypes). In certain embodiments, the biologically pure culture is at least 90, 91, 92, 93, 94, 95, 96, 97, 98, 99, 99.5, 99.6, 99.7, 99.8, or 99.9% pure (i.e., at least 90, 91, 92, 93, 94, 95, 96, 97, 98, 99, 99.5, 99.6, 99.7, 99.8, or 99.9% of the subcultures taken therefrom exhibit identical genotypes and phenotypes).

As used herein, the terms “fermenting”, “fermentation”, and grammatical variations thereof mean the production of alcohol and carbon dioxide from the breakdown of carbohydrate by yeast.

As used herein, the terms “maturing”, “maturation”, and grammatical variations thereof mean the time following the fermentation when the yeast can continue to affect the flavor and aroma of the beverages through their metabolic activity. Other interchangeable terms for this process include “conditioning” or “secondary fermentation.”

As used herein, the term “mash” is understood to mean a mix of milled grains and water used in brewing and distilling processes to produce wort. Typically, malted grains are heated in order to breakdown the starch in the grains into simple sugars, which can then be fermented in order to produce alcohol.

As used herein, the term “wort” is understood to mean the liquid extracted from the mashing process during a brewing or distilling process. Wort contains the sugars extracted from the malted grains which will be fermented by the brewing yeast in order to produce alcohol.

As used herein, the term “sour” in a fermented beverage refers to an acid, bitter, and/or sharp taste in the palate caused by the beverage. In certain embodiments, the sour taste is associated with the low pH of the fermented beverage.

As used herein, the term “low pH” in a fermented beverage indicates that the pH of the beverage is higher than or equal to about 2.5, and lower than or equal to about 4.5.

Beer styles have diverged over the past two hundred years into two main categories, ale and lager. Typically, ale is fermented at room temperatures (18° C.-24° C.) by S. cerevisiae with short maturation times (typically 10-30 days) and the beer characterized by some residual sweetness and moderate to high bitterness. In contrast, lagers are fermented at cellar temperatures (8° C.-14° C.) by S. pastorianus with long maturation times (4-12 weeks), and the beer characterized by minimal residual sweetness and low to moderate bitterness.

Sour-style beer (e.g., lambics) is produced by mixed culture fermentations of wort that, in addition to barley malt, usually contains some wheat and/or fruit and aged (oxidized) hops. The culture responsible for the fermentation is often based on the local cultures found within the specific brewery environment and may be composed of species from the genera of Saccharomyces, Brettanomyces, Dekkera, Kloeckera, Pediococcus, and Lactobacillus. The fermentations for sour-style beers are for extended times, typically several months to 2 years for traditional lambics. This type of beer is characterized by high levels of organic acids (mainly lactic and acetic with lesser amounts of propionic, isobutyric and butyric) with a pH ranging between 3.3 to 3.9. Ethyl acetate is also found at elevated levels in these beers, adding to the vinegar odor. The unique composition of the wort, combined with the variety and types of microbes used as well as the long fermentation times, results in very different flavor profiles from that of standard ales and lagers.

The present disclosure is directed to yeast species and strains newly identified for use in preparing fermented beverages, such as beer, thereby providing the brewer the ability to produce fermented beverages having new and distinctive flavor and aroma characteristics as compared to fermented beverages produced using the standard yeast species. In certain embodiments, a yeast useful for producing a fermented beverage can be Lachancea fermentati. In certain embodiments, the yeast can be a strain of Lachancea fermentati. In certain embodiments, the Lachancea fermentati strain can be Lachancea fermentati strain SWN001 deposited as ATCC Accession No. PTA-127803. In certain embodiments, the Lachancea fermentati strain comprises the nucleotide sequence of SEQ ID NO: 1 within the ITS region.

The disclosure provides a method of producing a fermented beverage, comprising fermenting at least one carbohydrate in the presence of Lachancea fermentati under suitable conditions for a period of time sufficient to produce a fermented beverage.

In certain embodiments, the disclosure provides a method of producing a fermented beverage, comprising fermenting at least one carbohydrate (primary fermentation) in the presence of Lachancea fermentati under suitable conditions for a period of time sufficient to produce a primary fermentation product; and maturing the primary fermentation product (secondary fermentation) to produce a fermented beverage.

In certain embodiments, a method of producing a fermented beverage is provided, comprising fermenting at least one carbohydrate to produce a primary fermentation product; and maturing the primary fermentation product in the presence of Lachancea fermentati under suitable conditions for a period of time sufficient to produce a fermented beverage. Thus, in certain embodiments, other yeast species (e.g., traditional yeast species such as Saccharomyces spp.) can be used in fermenting, and Lachancea fermentati can be used for maturing the primary fermentation product.

In certain embodiments, the Lachancea fermentati strain can be Lachancea fermentati strain SWN001 deposited as ATCC Accession No. PTA-127803. Thus, in certain embodiments, a method of producing a fermented beverage is provided, comprising fermenting at least one carbohydrate in the presence of Lachancea fermentati strain SWN001 under suitable conditions for a period of time sufficient to produce a fermented beverage. In certain embodiments, a method of producing a fermented beverage is provided, comprising fermenting at least one carbohydrate (primary fermentation) in the presence of Lachancea fermentati strain SWN001 under suitable conditions for a period of time sufficient to produce a primary fermentation product; and maturing the primary fermentation product (secondary fermentation) to produce a fermented beverage. In certain embodiments, a method of producing a fermented beverage is provided, comprising fermenting at least one carbohydrate to produce a primary fermentation product; and maturing the primary fermentation product in the presence of Lachancea fermentati strain SWN001 under suitable conditions for a period of time sufficient to produce a fermented beverage, wherein other yeast species (e.g., Saccharomyces spp.) can be used in fermenting, and Lachancea fermentati strain SWN001 can be used for maturing the primary fermentation product.

In certain embodiments, a combination of yeasts may be used in the fermenting and/or maturing steps. Thus, in certain embodiments, a method is provided comprising fermenting at least one carbohydrate in the presence of yeast, wherein the yeast comprises, consists essentially of, or consists of Lachancea fermentati strain SWN001, and may further comprise at least one additional yeast strain, to produce a primary fermentation product; and maturing the primary fermentation product (secondary fermentation) to produce a fermented beverage. In certain embodiments, a method of producing a fermented beverage is provided, comprising fermenting at least one carbohydrate to produce a primary fermentation product; and maturing the primary fermentation product in the presence of yeast, wherein the yeast comprises, consists essentially of, or consists of Lachancea fermentati strain SWN001, and further comprises at least one additional yeast strain, to produce a fermented beverage.

In certain embodiments, the at least one additional yeast strain comprises Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus, Hanseniaspora uvarum, Hanseniaspora guilliermondii, Hanseniaspora osmophila, Hanseniaspora vineae, Hansenula anomala, Issatchenkia occidentalis, Issatchenkia orientalis, Pichia kluyveri, Pichia caribbica, Pichia fermentans, Pichia kudriavzevii, Pichia membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae, Wickerhamomyces subpelliculosus, Wickerhamomyces anomalus, Zygosaccharomyces rouxii, Zygosaccharomyces bailii, Zygosaccharomyces fermentati, Zygosaccharomyces florentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

In certain embodiments, a fermented beverage can be beer, including but not limited to, ale or lager. In certain embodiments, the ale or lager can be sour-style beer. In certain embodiments, the ale can be sour-style beer. In certain embodiments, the lager can be sour-style beer (e.g., when the fermentation occurs at a low temperature). In certain embodiments, the fermented beverage can be alcoholic cider. In certain embodiments, the fermented beverage can be mead (e.g., fermenting honey). In certain embodiments, the fermented beverage can be wine. Methods for brewing these and other different types of fermented beverages are known in the art.

The fermented beverage brewing process comprises, in general, malting, mashing, lautering, boiling, fermenting, maturing (conditioning), filtering and packaging. As understood by those of skill in art, some of these steps can be skipped and others added, as well as steps can be modified to achieve the particular desired fermented beverage.

Fermentation to produce a beverage can comprise (1) a primary fermentation in which the yeast ferment the at least one carbohydrate selected by the brewer to produce a primary fermentation product, and (2) a maturing step (i.e., secondary fermentation, conditioning or aging), which allows the fermentation process to continue acting on the primary fermentation product, typically in the presence of a reduced and/or modified yeast population. The maturing process can be carried out under the same or different conditions (e.g., temperature) than that used for the fermenting step. Thus, in certain embodiments, a change in the temperature as compared to the primary fermentation step can be an increase or decrease in temperature. Furthermore, in certain embodiments, the production of a fermented beverage can comprise a primary fermenting step but not a maturing step. Instead, the process may be complete and the fermented beverage produced after the fermenting step.

In certain embodiments, the step of fermenting at least one carbohydrate may be carried out for about 1 day to about 30 days (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days and the like, or any range or value therein). In certain embodiments, the step of fermenting at least one carbohydrate may be carried out for about 2 days to about 30 days (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30 days and the like, or any range or value therein). In certain embodiments, the step of fermenting at least one carbohydrate may be carried out for about 2 days to about 21 days (e.g., about 2, 3, 4, 5, 6, 7, 8, 9, 10, 11, 12, 13, 14, 15, 16, 17, 18, 19, 20, 21 days and the like, or any range or value therein).

In certain embodiments, the step of fermenting carbohydrates may be carried out at a temperature of about 4° C. to about 24° C. (e.g., about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C. and the like, or any range or value therein). In certain embodiments, the step of fermenting at least one carbohydrate may be carried out at temperature of about 8° C. to about 14° C. or about 18° C. to about 24° C. Any combination of temperature and time for fermenting the at least one carbohydrate can be chosen by the brewer.

Similar to the choice of time and temperature for fermenting at least one carbohydrate, the selection of temperature and time for maturing the primary fermentation product depends on the type of beer and the characteristics of the beer desired by the brewer.

For example, when producing lagers, generally the temperature is reduced in the maturing step as compared to the fermenting step. When producing ales, the temperature chosen for maturing may be about the same or may be lower than that for the fermenting step and for sour-style beers, the temperature for maturing can be highly variable. Thus, in some embodiments, maturing may be carried out at a temperature of about 1° C. to about 24° C. (e.g., about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C. and the like, or any range or value therein).

In certain embodiments, the time period for maturing can be from about 10 minutes to about 16 weeks (e.g., 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 35 min, 40 min, 45 min, 50 min, 55 min, 1 hr, 1.5 hr, 2 hr, 2.5 hr, 3 hr, 3.5 hr, 4 hr, 4.5 hr, 5 hr, 5.5 hr, 6 hr, 6.5 hr, 7 hr, 7.5 hr, 8 h, 9 hr, 10 hr, 11 hr, 12 hr, 15 hr, 18 hr, 22 hr, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks and the like, or any range or value therein). In certain embodiments, the time period for maturing can be from about 2 days to 21 days (3 weeks) (e.g., about 2, 3, 4, 5, 6, 7, 8, 9, 10, 11, 12, 13, 14, 15, 16, 17, 18, 19, 20, 21 days and the like, or any range or value therein), or about 10 days to about 12 weeks (e.g., about 10 days, 11 days, 12 days, 13 days, 2 weeks, 3 weeks, 4 weeks, 5 weeks, 6 weeks, 7 weeks, 8 weeks, 9 weeks, 10 weeks, 11 weeks, 12 weeks and any range or value therein). In certain embodiments, the time period for maturing can be up to about 3 months. In certain embodiments, the time period for maturing a sour-style beer and/or an ale can be, for example, for about 2 days to about 30 days (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or any range or value therein). In certain embodiments, the time period for maturing can be about 4 weeks to about 12 weeks, for example, for maturing a lager beer. As the skilled artisan would be aware, a brewer can select any combination of temperature and time for maturing the primary fermentation product with the choice being determined by the desired characteristics for the fermented beverage being brewed (e.g., aroma, flavor, foam stability, acidity, alcohol content and mouth feel).

Notably, the use of Lachancea fermentati in the fermenting and/or maturing steps of this disclosure allows the production of a sour-style beer in much less time than is typical for producing these types of fermented beverages. Thus, in certain embodiments, a sour-style beer of the present disclosure can be made in about 3 days to about 12 weeks (e.g., about 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, or any range or value therein). In contrast, using yeast and bacterial species standard in the art (e.g., Saccharomyces spp., Brettanomyces spp., Dekkera spp., Kloeckera spp., Pediococcus spp, and Lactobacillus spp.), the time period needed to produce a sour-style beer is about 6 months to about 2 years.

The source of the at least one carbohydrate for fermenting in the process of producing a fermented beverage is an important determinant in the strength and flavor of the resultant beverage. Any carbohydrate source that can be fermented by the yeast that produces the desired fermented beverage characteristics can be used. In some embodiments, the at least one carbohydrate can be any starch source. In some embodiments, the at least one carbohydrates can be grain, including, but not limited to, barley, rye, wheat, corn, sorghum, rice, or any combination thereof. In some embodiments, the at least one carbohydrate can be a vegetable, for example, a root vegetable, including, but not limited to, potato, cassava, sugar beet, or any combination thereof. In some embodiments, the at least one carbohydrate can be any fruit and/or fruit juice containing fructose, glucose and/or sucrose including, but not limited to, apple, strawberry, black currant, cherry, raspberry, blackberry, blueberry, citrus (e.g., orange, lemon). In certain embodiments, when fruit juice is the at least one carbohydrate, the fruit juice does not comprise added preservatives (e.g., sodium benzoate). In certain embodiments, the fruit juice can be unfiltered, organic, and/or pasteurized. In certain embodiments, the at least one carbohydrate can be starch, malt, wort, corn syrup, sugar cane, molasses, honey, and/or any combination thereof. Of course, any combination of carbohydrate sources can be used in the fermenting step, including, but not limited to, combinations of any grain, vegetable, malt, wort, corn syrup, sugar cane, fruit and/or fruit juice, honey, and/or molasses. Exemplary malts include 2-row, Black Patent, English Pale, Scottish Crystal, Cara Vienne, Pilsner, Special Roast, Caramel, Carapils, Chocolate, Victory, Vienna, Munich, Weizen and/or any combination thereof.

In certain embodiments, the method of producing a fermented beverage further comprises adding at least one (i.e., one or more, 1, 2, 3, 4, 5, 6, 7, 8, 9, 10, or more) flavoring in any step prior to fermenting, during and/or after fermenting in the methods of producing a fermented beverage. Any flavoring or combination of flavorings may be used that impart the flavor and aroma desired by the brewer. Further, when more than one flavoring is added, some may be added prior to fermenting and some may be added after fermenting. In certain embodiments, a flavoring can include, but is not limited to, flowers (e.g., hops, hibiscus, rose, lavender), fruit (e.g., puree and/or extract of apple, pear, peach, apricot, plum, orange, cherry, raspberry, strawberry, banana, and the like), spices (e.g., coriander, ginger, vanilla, citrus peel, nutmeg, allspice, peppercorn), nuts (e.g., hazelnut), chocolate, honey, rose water and the like, and/or any combination thereof.

In certain embodiments, the at least one flavoring can be hops. Any type of hops can be used and the choice will depend on the desired characteristics (e.g., bittering, aroma and flavor) of the fermented beverage being brewed. In some embodiments, the hops can include, but are not limited to, Admiral, Ahtanum, Amarillo, Apollo, Aramis, Bor, Bramling Cross, Bravo, Brewer's Gold, Bullion, Calypso, Cascade, Centennial, Challenger, Chelan, Chinook, Citra, Cluster, Columbus, Comet, Crystal, El Dorado, Eroica, Feux-Coeur Francais, First Gold, Fuggles, Galaxy, Galena, Glacier, Goldings, Green Bullet, Greenburg, Hallertau, Hallertauer Herkules, Hallertauer Magnum, Hallertauer Taurus, Herald, Hersbrucker, Horizon, Junga, Kohatu, Liberty, Lublin, Magnum, Marynka, Merkur, Millennium, Motueka, Mount Hood, Mount Rainier, Nelson Sauvin, Newport, Northdown, Northern Brewer, Nugget, Opal, Pacific Gem, Pacific Jade, Pacifica, Palisade, Perle, Phoenix, Pilgrim, Pilot, Pioneer, Polnischer Lublin, Premiant, Pride of Ringwood, Progress, Rakau, Riwaka, Saaz, San Juan Ruby Red, Santiam, Saphir, Satus, Select, Simcoe, Sladek Sorachi Ace, Smaragd, Sonnet Golding, Sorachi Ace, Southern Cross, Spalt, Stella, Sterling, Sticklebract, Strisselspalt, Styrian Atlas, Styrian Aurora, Styrian Bobek, Styrian Celeia, Styrian Goldings, Summer Saaz, Summit, Super Alpha, Super Galena, Super Pride, Sybilla, Tardif de Bourgogne, Target, Tettnang, Tillicum, Tomahawk, Topaz, Tradition, Ultra, Vanguard, Wai-iti, Warrior, Whitbread Golding Variety, Willamette, Zeus, Zythus, and/or any combination thereof.

The present disclosure also provides a fermented beverage produced by any method of this disclosure. In certain embodiments, the fermented beverage of the disclosure is beer. In certain embodiments, the fermented beverage of the disclosure is lager. In certain embodiments, the fermented beverage is ale. In further embodiments, the fermented beverage is sour-style beer comprising a pH of about 3.0 to about 4.5. In certain embodiments, a sour-style beer of the disclosure comprises lactic acid and acetic acid produced during the brewing process (that is, lactic acid is not added). In certain embodiments, a sour-style beer of the invention comprises esters, acetate esters, ethyl acetate, isoamyl acetate, phenylethyl acetate, medium chain fatty acid ethyl esters, ethyl hexanoate, ethyl octanoate, or any combination thereof. In certain embodiments, the fermented beverage can be alcoholic cider. In certain embodiments, the fermented beverage can be mead. In certain embodiments, the fermented beverage can be wine.

The disclosure provides a kit comprising at least one Lachancea fermentati strain contemplated herein and one or more items or ingredients suitable to produce fermented beverage. The disclosure further provides a kit comprising Lachancea fermentati strain SWN001 deposited as ATCC Accession No. PTA-127803, and one or more ingredients suitable to produce a fermented beverage. The disclosure further provides a kit comprising a Lachancea fermentati strain, wherein the Lachancea fermentati strain comprises the nucleotide sequence of SEQ ID NO: 1 within the ITS region, and one or more items or ingredients suitable to produce a fermented beverage.

Illustrative ingredients (e.g., carbohydrates, flavoring agents) suitable to produce a fermented beverage are described elsewhere herein.

In certain embodiments, the kit comprises packaged Lachancea fermentati strain SWN001. In certain embodiments, the kit comprises dried Lachancea fermentati strain SWN001 in the form of a powder. In certain embodiments, the kit comprises Lachancea fermentati strain SWN001 in a vacuum sealed container. In certain embodiments, the kit comprises Lachancea fermentati strain SWN001 from a fresh propagation or a post-fermentation slurry in a vented container.

In certain embodiments, the kit further comprises one or more items necessary for performing the methods of the disclosure selected from one or more vessels adapted and configured for wort fermentation, one or more thermometers, one or more hydrometers, one or more vessels adapted and configured for wort boiling, one or more vessels adapted and configured for pasteurization, one or more vessels adapted and configured for beer storage, one or more vessels adapted and configured for aging of beer. In other embodiments, the kit further comprises one or more items selected from compressed CO₂ tanks, compressed N₂ tanks, gas regulators, tubing, and pressure gauges.

In certain embodiments, the kit further comprises instructional materials containing instructions for performing the methods of the disclosure. In certain embodiments, the instructional materials provide information pertaining to brewing beer with Lachancea fermentati strain SWN001 using the ingredients of the kit of the disclosure.

As used herein, the term “instructional material” includes a publication, a recording, a diagram, or any other medium of expression that can be used to communicate the usefulness of the composition of the disclosure in a kit. The instructional material of the kit may, for example, be affixed to a container that contains the composition of the disclosure or be shipped together with a container that contains the composition. Alternatively, the instructional material may be shipped separately from the container with the intention that the recipient uses the instructional material and the composition cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The following numbered embodiments also form part of the present disclosure:
1. A method of producing a fermented beverage, comprising: fermenting at least one carbohydrate in the presence of a Lachancea fermentati strain to produce a fermented beverage.
2. The method of embodiment 1, wherein the at least one carbohydrate comprises starch, grain, vegetable, malt, wort, corn syrup, sugar cane, molasses, honey, fruit, fruit juice, or a combination thereof.
3. The method of embodiment 1 or embodiment 2, wherein the at least one carbohydrate comprises wort.
4. The method of any one of embodiments 1-3, further comprising adding at least one flavoring agent prior to fermenting or after fermenting.
5. The method of embodiment 4, wherein the flavoring agent comprises hops.
6. The method of any one of embodiments 1-5, wherein the at least one carbohydrate is fermented in the absence of a lactic acid producing bacteria.
7. The method of any one of embodiments 1-6, further comprising adding glucose prior to fermenting or after fermenting.
8. The method of any one of embodiments 1-7, wherein the fermented beverage is beer.
9. The method of embodiment 8, wherein the beer is a sour-style beer.
10. The method of any one of embodiments 1-9, wherein the Lachancea fermentati strain comprises the nucleotide sequence of SEQ ID NO: 1 within the ITS region.
11. The method of any one of embodiments 1-10, wherein the Lachancea fermentati strain is Lachancea fermentati strain SWN001 deposited as ATCC Accession No. PTA-127803.
12. A fermented beverage produced by the method of any one of embodiments 1-11.
13. A biologically pure culture of Lachancea fermentati strain SWN001 deposited as ATCC Accession No. PTA-127803.
14. The biologically pure culture of embodiment 13, wherein the Lachancea fermentati strain comprises the nucleotide sequence of SEQ ID NO: 1 within the ITS region.
15. A kit comprising: a Lachancea fermentati strain; and one or more ingredients to produce a fermented beverage.
16. The kit of embodiment 15, wherein the one or more ingredients comprises at least one carbohydrate or at least one flavoring agent.
17. The kit of embodiment 16, wherein the at least one carbohydrate comprises starch, grain, vegetable, malt, wort, corn syrup, sugar cane, molasses, honey, fruit, fruit juice, glucose, or a combination thereof.
18. The kit of embodiment 16 or embodiment 17, wherein the flavoring agent comprises hops.
19. The kit of any one of embodiments 15-18, further comprising instructional materials for producing a fermented beverage.
20. The kit of any one of embodiments 15-19, wherein the Lachancea fermentati strain comprises the nucleotide sequence of SEQ ID NO: 1 within the ITS region.
21. The kit of any one of embodiments 15-20, wherein the Lachancea fermentati strain is Lachancea fermentati strain SWN001 deposited as ATCC Accession No. PTA-127803
22. Use of the Lachancea fermentati strain SWN001 deposited as ATCC Accession No. PTA-127803 or the kit of any one of embodiments 15-21 to produce a fermented beverage.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Strain Isolation

The objective of this example was to identify wild yeast strains capable of producing beer with unique organoleptic qualities. To this end, 100 samples from various flora and fauna were collected at a Christmas tree farm in Oskaloosa, Iowa. These samples were then cultured in 5 mL of YPD media. Observations for fermentation activities, particularly carbon dioxide production, were made, leading to the plating of active samples on YPD-agar. The plates underwent visual analysis, and potential yeast colonies were examined under a microscope. Identified yeast colonies were subsequently cultured on YPD-agar. From these, single colonies were selected to inoculate 20 mL of YPD, resulting in the growth of new yeast cultures. These cultures were subjected to aroma analysis, with those emitting pleasant aromas being further tested for carbohydrate utilization and ethanol resistance. Emphasis was placed on strains that could ferment maltose and exhibited moderate ethanol resistance. Selected yeasts underwent genotyping through PCR amplification of the ITS region, followed by sequencing of the PCR products. This process identified four variants of the species Lachancea fermentati. Notably, one variant (ISU-Lf #14) exhibited superior fermentation kinetics, effective flocculation properties, and enhanced souring capability, presenting a promising candidate for single-microorganism sour beer production. This strain was designated as SWN001.

SEQ ID NO: 1 is the sequence of the ITS region of Lachancea fermentati strain SWN001:

gggaaaaatggaaggaaggcagccgggaaaggtcagacgcctgcgcttaa
ttgcgcggccgaagatactttctgttaacgactgctctgctacacacaca
ctttggagtagttttttacaacacttcttctttgggcttcggcccagagg
ttacaaacacaaacaacttttgtattatatcatagtcaagaatttttcat
tagaaaaaaatattcaaaactttcaacaacggatctcttggttctcgcat
cgatgaagaacgcagcgaattgcgataagtattgtgaattgcagattttc
gtgaatcatcgaatctttgaacgcacattgcgccctctggtattccagag
ggcatgcctgtttgagcgtcatttccttctcaaaccttagggtttggtag
tgagtgatactctttctagggttaacttgaaaatgctggccatctggctg
ttgctggctgaggctttagtccagtctgctgatactctgcgtattaggtt
ttaccaactcgtgggggcttgagcggacgctacaaagacttttgctaaaa
gtacagacaacctggcgaacagtattcactaagtttgacctcaaatcagg
taggattacccgctgaacttaagcatatcatag

### Example 2: Strain Performance

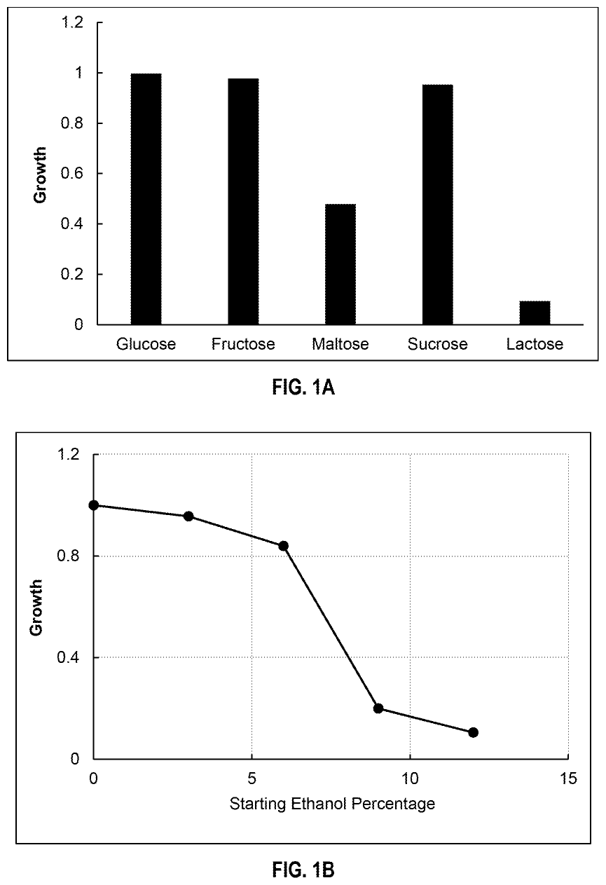

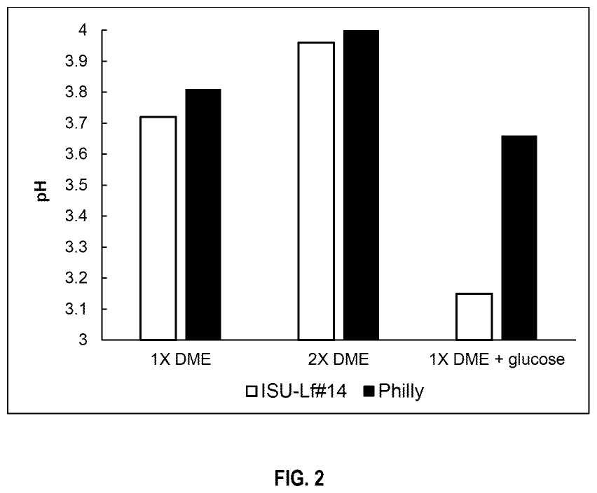

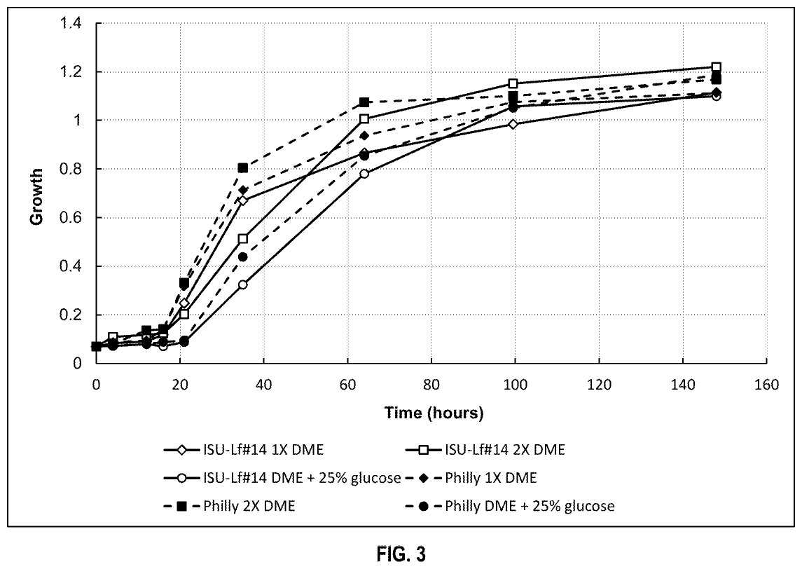

Beer produced with ISU-Lf #14 yeast was tart/sour. The ISU-Lf #14 yeast has acceptable carbohydrate utilization and ethanol tolerance for the production of beer, hard cider, and low alcohol fruit wines (FIG. 1A-B). ISU-Lf #14 has superior souring capabilities compared to the commercial yeast species Lachancea thermotolerans (known as Philly Sour and sold by Lallemand Brewing). Also, it is notable that the pH can be controlled by the addition of glucose to the fermentation media (FIG. 2). This provides a unique way to control the sourness of the resulting beer. The growth kinetics of ISU-Lf #14 is very similar to the Philly Sour yeast (FIG. 3).

### Deposit of Biological Material

Lachancea fermentati strain SWN001 was deposited on Sep. 11, 2024, under the provisions of the Budapest Treaty with the American Type Culture Collection (ATCC) at 10801 University Boulevard, Manassas, VA 20110-2209, and has been assigned Accession No. PTA-127803. The deposit will irrevocably and without restriction or condition be available to the public upon issuance of a patent. This deposit will be maintained under the terms of the Budapest Treaty on the International Recognition of the Deposit of Microorganisms for the Purposes of Patent Procedure. This deposit was made merely as a convenience for those of skill in the art and are not an admission that a deposit is required under 35 U.S.C. § 112. However, it should be understood that the availability of a deposit does not constitute a license to practice the subject invention in derogation of patent rights granted by government action. The deposit will be maintained without restriction in the ATCC Depository, which is a public depository, for a period of 30 years, or 5 years after the most recent request, or for the enforceable life of the patent, whichever is longer, and will be replaced if it ever becomes nonviable during that period.

What is claimed is:

1. A method of producing a fermented beverage, comprising:
fermenting at least one carbohydrate in the presence of a Lachancea fermentati strain SWN001 deposited as ATCC Accession No. PTA-127803 to produce a fermented beverage.

2. The method of claim 1, wherein the at least one carbohydrate comprises starch, grain, vegetable, malt, wort, corn syrup, sugar cane, molasses, honey, fruit, fruit juice, or a combination thereof.

3. The method of claim 1, wherein the at least one carbohydrate comprises wort.

4. The method of claim 1, further comprising adding at least one flavoring agent prior to fermenting or after fermenting.

5. The method of claim 4, wherein the flavoring agent comprises hops.

6. The method of claim 1, wherein the at least one carbohydrate is fermented in the absence of a lactic acid producing bacteria.

7. The method of claim 1, further comprising adding glucose prior to fermenting or after fermenting.

8. The method of claim 1, wherein the fermented beverage is beer.

9. The method of claim 8, wherein the beer is a sour-style beer.

10. The method of claim 1, wherein the Lachancea fermentati strain comprises the nucleotide sequence of SEQ ID NO: 1 within the ITS region.

11. A fermented beverage produced by the method of claim 1.

12. A biologically pure culture of Lachancea fermentati strain SWN001 deposited as ATCC Accession No. PTA-127803.

13. The biologically pure culture of claim 12, wherein the Lachancea fermentati strain comprises the nucleotide sequence of SEQ ID NO: 1 within the ITS region.

14. A kit comprising:
Lachancea fermentati strain SWN001 deposited as ATCC Accession No. PTA-127803; and
one or more ingredients to produce a fermented beverage.

15. The kit of claim 14, wherein the one or more ingredients comprises at least one carbohydrate or at least one flavoring agent.

16. The kit of claim 15, wherein the at least one carbohydrate comprises starch, grain, vegetable, malt, wort, corn syrup, sugar cane, molasses, honey, fruit, fruit juice, glucose, or a combination thereof.

17. The kit of claim 15, wherein the flavoring agent comprises hops.

18. The kit of claim 14, further comprising instructional materials for producing a fermented beverage.

19. The kit of claim 14, wherein the Lachancea fermentati strain comprises the nucleotide sequence of SEQ ID NO: 1 within the ITS region.


SEQUENCE LISTING (1 sequence)

SEQ ID NO: 1 - DNA, 633 bp, Lachancea fermentati
  source 1..633: mol_type = genomic DNA
gggaaaaatggaaggaaggcagccgggaaaggtcagacgcctgcgcttaattgcgcggccgaagatactttctgttaacgactgctctgctacacacacactttggagtagttttttacaacacttcttctttgggcttcggcccagaggttacaaacacaaacaacttttgtattatatcatagtcaagaatttttcattagaaaaaaatattcaaaactttcaacaacggatctcttggttctcgcatcgatgaagaacgcagcgaattgcgataagtattgtgaattgcagattttcgtgaatcatcgaatctttgaacgcacattgcgccctctggtattccagagggcatgcctgtttgagcgtcatttccttctcaaaccttagggtttggtagtgagtgatactctttctagggttaacttgaaaatgctggccatctggctgttgctggctgaggctttagtccagtctgctgatactctgcgtattaggttttaccaactcgtgggggcttgagcggacgctacaaagacttttgctaaaagtacagacaacctggcgaacagtattcactaagtttgacctcaaatcaggtaggattacccgctgaacttaagcatatcatag